(19) United States
(12) Patent Application Publication
(10) Pub. No.: US 2023/0368892 A1
(43) Pub. Date: Nov. 16, 2023

(54) METHOD FOR AUTOMATING RADIOLOGY WORKFLOW

(71) Applicant: GE Precision Healthcare LLC, Wauwatosa, WI (US)

(72) Inventors: Benoit Doreau, Meudon (FR); Diana Paiva Moreira Batista, Versailles (FR); Cedric Hermel, Villeloin-Coulange (FR)

(21) Appl. No.: 17/662,976

(22) Filed: May 11, 2022

(51) Int. Cl. G16H 30/20

(52) U.S. Cl. CPC G16H 30/20

(57) ABSTRACT

Methods and systems are provided for automating steps of a workflow within a medical image processing system. In one example, a method for a medical image processing system comprises extracting expressions from description fields of a set of Digital Imaging and Communications in Medicine (DICOM) files of a medical imaging exam that match reference terms of an ontology; mapping the matching reference terms of the ontology to one or more lexicon entries of a radiology lexicon; selecting a suitable software application to review the medical imaging exam based on the one or more lexicon entries; opening the suitable software application on a device of the medical image processing system; and displaying the medical imaging exam on a display of the device within the suitable software application. The ontology includes reference terms generated from DICOM sources, vocabulary from other relevant lexicons, ontologies, reference databases, and human experts.

### TECHNICAL FIELD

Embodiments of the subject matter disclosed herein relate to processing images generated during medical exams.

### BACKGROUND

In the radiology domain, users expect software applications to free them from repetitive and non-clinical decisions, so that they can focus on clinical tasks. More specifically, reviewing a radiology exam typically involves carrying out a set of preparation actions for creating the conditions for an efficient clinical review. The preparation actions represent decision points of a workflow, which may include, for example, selecting one or more exams, selecting series, selecting an application to launch, selecting a layout, selecting a tool, and the like. Carrying out the preparation actions may involve numerous interactions with a user interface (e.g., mouse clicks), which may take time and may increase a level of frustration of users. As relevant technologies evolve, users increasingly request and expect higher levels of automation from radiology applications, where artificial intelligence (AI) techniques are used to automate the preparation steps. A number of the preparation actions may be reduced if an automated assistance service can automatically determine or predict a desired configuration of an image processing system or application.

A set of Digital Imaging and Communications in Medicine (DICOM) files associated with a medical exam includes relevant information about images included in the medical exam, including an acquisition modality and acquisition protocol data, a study the images are part of, and image pixels. This information could be used by the automated assistance service to aid the user in selecting options at various steps of the workflow. Additionally, the DICOM information could be leveraged for other purposes, such as to index exams for storage and retrieval purposes, to route exams to appropriate radiologists, and/or to proactively launch algorithms on exams using batch processing systems. However, an availability and a reliability of the DICOM files can vary widely across manufacturers and models, and include text manually entered into description fields of the DICOM file in non-standardized ways. Because of a wide variety of terms, description styles, and languages used in the description fields, it may be difficult, time consuming, and costly for machine learning algorithms to efficiently map content of the description fields to a standard format in order to take automatic actions with respect to medical exams.

### SUMMARY

The current disclosure at least partially addresses one or more of the above identified issues by a method for a medical image processing system, the method comprising extracting expressions from description fields of a set of Digital Imaging and Communications in Medicine (DICOM) files of a medical imaging exam that match reference terms of an ontology; mapping the matching reference terms of the ontology to one or more lexicon entries of a radiology lexicon; selecting a suitable software application to review the medical imaging exam based on the one or more lexicon entries; opening the suitable software application on a device of the medical image processing system; and displaying the medical imaging exam on a display of the device within the suitable software application. The one or more lexicon entries may further be used to select a layout of the suitable software application and/or select an algorithm of the suitable software application to apply to the medical imaging exam.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1 is a schematic block diagram of a medical image processing system, in accordance with one or more embodiments of the present disclosure;

FIG. 2 is a schematic block diagram indicating a flow of data between an automated workflow assistance service and a workflow, in accordance with one or more embodiments of the present disclosure;

FIG. 3A is a figure showing example description fields of a DICOM file, as prior art;

FIG. 3B is a figure showing an exemplary set of expressions extracted from the description fields of FIG. 3A, in accordance with one or more embodiments of the present disclosure;

FIG. 3C is a figure showing an exemplary output of an automated workflow assistance service based on the set of expressions of FIG. 3B, in accordance with one or more embodiments of the present disclosure;

FIG. 4A is a flowchart illustrating an exemplary method for generating a prioritized list of lexicon entries that match expressions extracted from description fields of a set of DICOM files of a medical imaging exam, in accordance with one or more embodiments of the present disclosure;

FIG. 4B is a flowchart illustrating an exemplary method for using a prioritized list of lexicon entries associated with a medical imaging exam to automate one or more steps of a workflow of a user, in accordance with one or more embodiments of the present disclosure;

FIG. 5 is a flowchart illustrating an exemplary method for generating an ontology of a text processing model of an automated workflow assistance service, in accordance with one or more embodiments of the present disclosure;

FIG. 6 is a flowchart illustrating an exemplary method for enriching an ontology of a text processing model of an automated workflow assistance service, in accordance with one or more embodiments of the present disclosure;

FIG. 7 is a flowchart illustrating an exemplary method for enriching translated terms of an ontology based on a set of DICOM sources, in accordance with one or more embodiments of the present disclosure; and

FIG. 8 is a flowchart illustrating an exemplary method for evaluating a performance of an ontology of a text processing model of an automated workflow assistance service, in accordance with one or more embodiments of the present disclosure.

### DETAILED DESCRIPTION

The methods and systems described herein relate to using descriptive data associated with a medical imaging exam to automate one or more actions performed on the medical imaging exam, for example, by a user (e.g., a radiologist) reviewing the medical imaging exam via one or more software applications running on a computing device, or by a computer system. With respect to medical images acquired during a radiology exam, the descriptive data may be represented and/or stored in accordance with a Digital Imaging and Communications in Medicine (DICOM) standard. The DICOM standard defines a file format that includes description fields for information that an image processing application may use to display and/or preprocess imaging data included in a DICOM file.

For example, a user reviewing radiology exams via an image processing system may have a workflow that periodically involves decision points, where a user selects one option for a next workflow step from a list of possible options. For example, when initiating a medical exam review, at a first decision point, the user may have to select a relevant application to review the medical exam from a list of applications. When the list of applications is long, selecting a suitable application can be frustrating for the user. A suitable application may be based on a type of the medical imaging exam (e.g., a type of image acquisition), which may be determined from description field data of a DICOM file of the medical exam. If the type of medical imaging exam can be determined, a suitable image processing application may be automatically selected for performing the exam. Thus, in order to improve operational efficiency and workflow control, an artificial intelligence (AI) agent such as an automated decision assistance service may be used by an image processing system to automatically determine the type of a medical exam based on the DICOM description field data, and use the type to select the suitable application.

However, while in principle the standardization brought by DICOM should make it easy to determine parameters that describe a medical imaging exam, in practice, a use of DICOM fields may vary substantially from one manufacturer to another, and/or across models from the same manufacturer. Key concepts and/or dimensions, such as the anatomy and the use of contrast, may not be reliably captured in the DICOM fields that are defined for a particular use. Some of the DICOM fields may be optional, or may be diversely fed depending on a manufacturer, model, and/or instance of an acquisition system.

Because there may be dozens of DICOM fields available for each series of the exam, one approach to automatically determining which applications are suitable and/or which application should be launched entails training a machine learning model on a huge number of examples collected across various systems. However, collecting a sufficient number of examples may be costly and time consuming. Additionally, a time taken to run the model during selection of a suitable application may exceed a threshold duration, generating delays that make the model unfeasible for use in real time.

Thus, a novel approach is proposed herein for characterizing a medical imaging exam based on extracting expressions from the DICOM description fields of the medical imaging exam, and matching the expressions to relevant terms of an ontology. The ontology may be pre-defined based on information from a selection of DICOM sources and enriched based on information extracted from other ontologies in the domain, including ontologies in other languages. In various embodiments, the ontology may be manually curated. The relevant terms of the ontology may then be mapped to a unidimensional type of the medical imaging exam. For example, in some embodiments, the unidimensional type of the medical imaging exam may correspond to a matching entry of a radiology lexicon, such as the RadLex lexicon.

The unidimensional type may then be used to determine a probable option for the suitable application to review the medical exam. If a probable option is found, the automated decision assistance service may automatically select the option. If no probable option is detected, the service may propose one or more options (of a comprehensive list of available options) to the user to select from. The user may select a desired option from the list more rapidly than selecting an option from the comprehensive list of available options. In this way, an amount of time taken to select the suitable application may be reduced, leading to a faster, easier, and more efficient review and a more desirable user experience.

While the methods and systems disclosed herein are described largely in reference to a workflow automation example, it should be appreciated that the unidimensional types may be used by other types of AI agents of various healthcare systems and networks to automate various actions that may be performed on medical imaging exams. For example, a routing system may route a medical exam to a radiologist for review based on the unidimensional type. The unidimensional types may be used to index medical imaging exams for efficient retrieval from databases of the healthcare systems. An algorithm of a batch processing system may apply to one or more medical exams, where the one or more medical exams may be selected based on the unidimensional types. In other embodiments, a medical imaging exam may be mapped to one or more regulatory categories based on the unidimensional types, for reporting purposes, such as to select a reporting template, or the unidimensional types may be used for operational efficiency analytics. Libraries of exams may be generated based on unidimensional types for training machine learning algorithms. Additionally or alternatively, the unidimensional types may be used for categorizing exams or image series data for post-market analysis by sales and marketing teams, or a sales targeting model may be built based on the unidimensional types to identify a set of relevant options to propose to a prospective client. Software applications may use the unidimensional types for cache management, where some types of imaging exams may be more likely to be reopened in a given time frame than others. It should be appreciated that the examples provided herein are for illustrative purposes, and the unidimensional types generated from DICOM description data as described herein may be used by other systems and/or for other purposes without departing from the scope of this disclosure.

A further advantage of the systems and methods disclosed herein is that since DICOM description field data is independent of an acquisition system's vendor and/or model, by leveraging the DICOM description field data, the unidimensional types may be used with data produced by various proprietary systems. As a result of this system and model independence, it is possible to use data from multiple systems and sites to learn which workflow behavior is best suited for a given unidimensional type. The workflow behaviors may be visible and/or editable by users, which may not be possible for systems that rely on higher dimensional input.

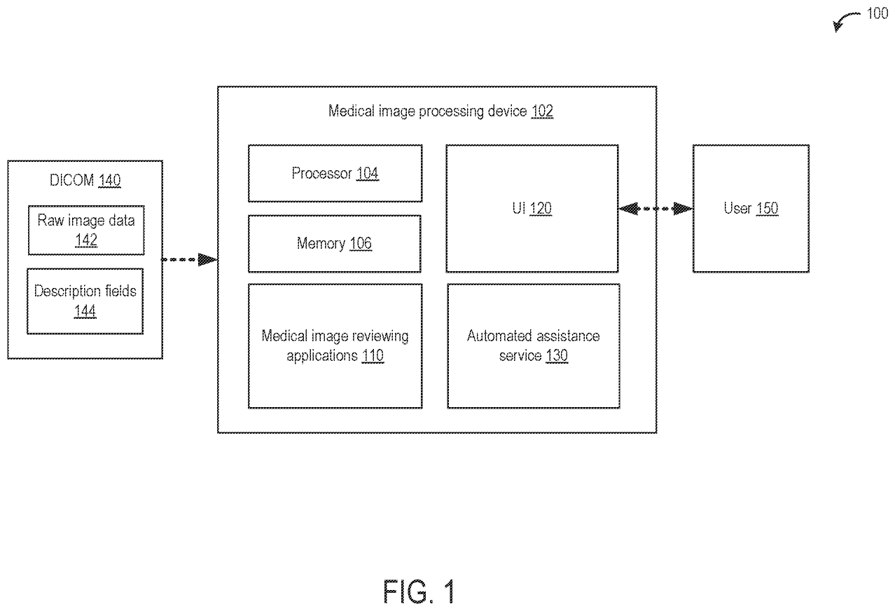

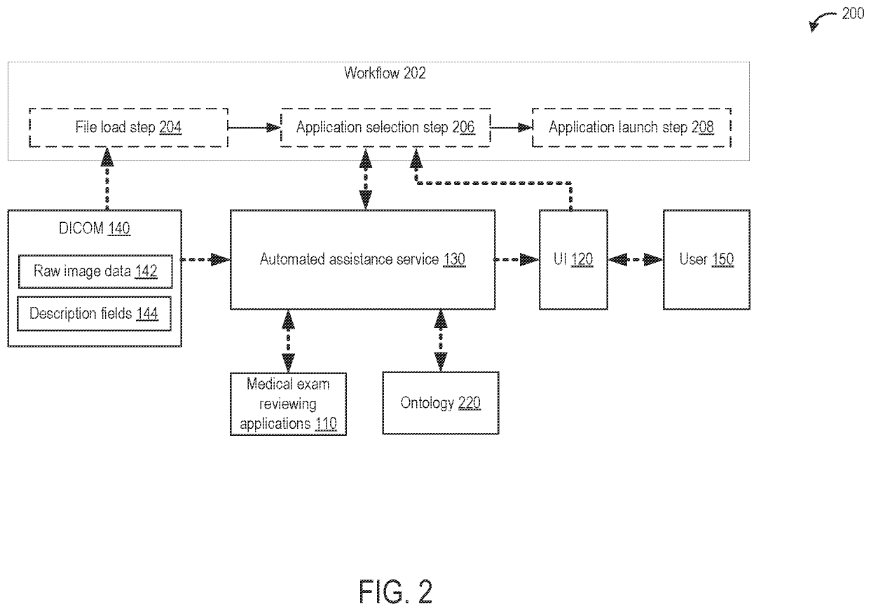

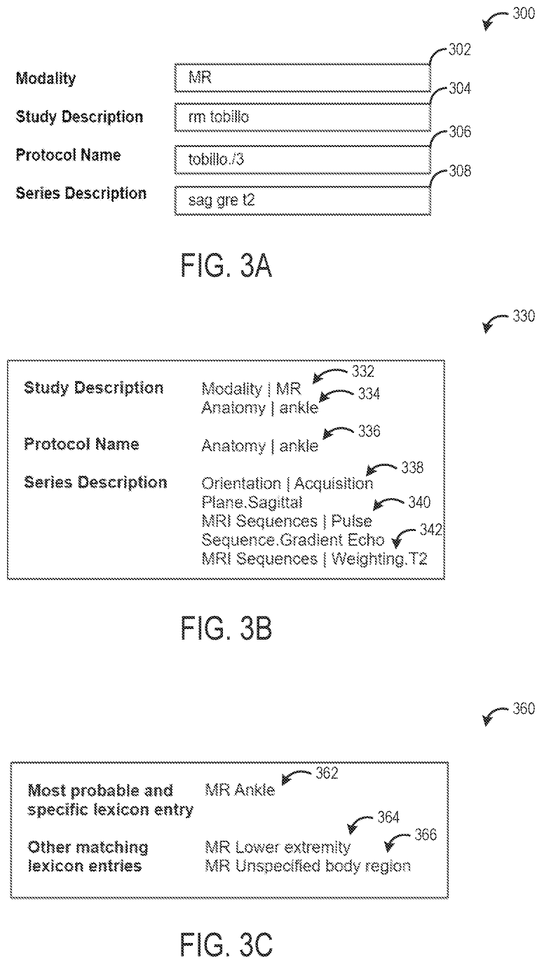

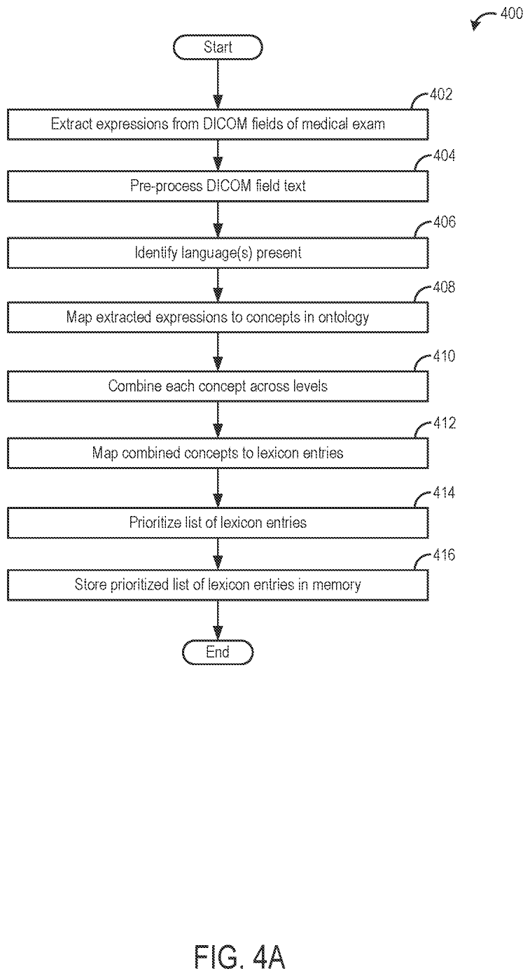

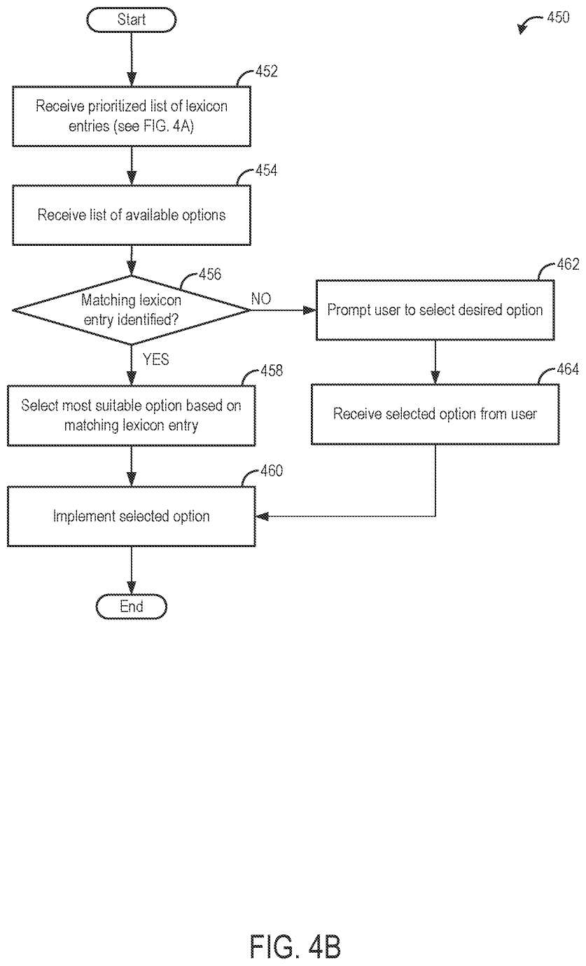

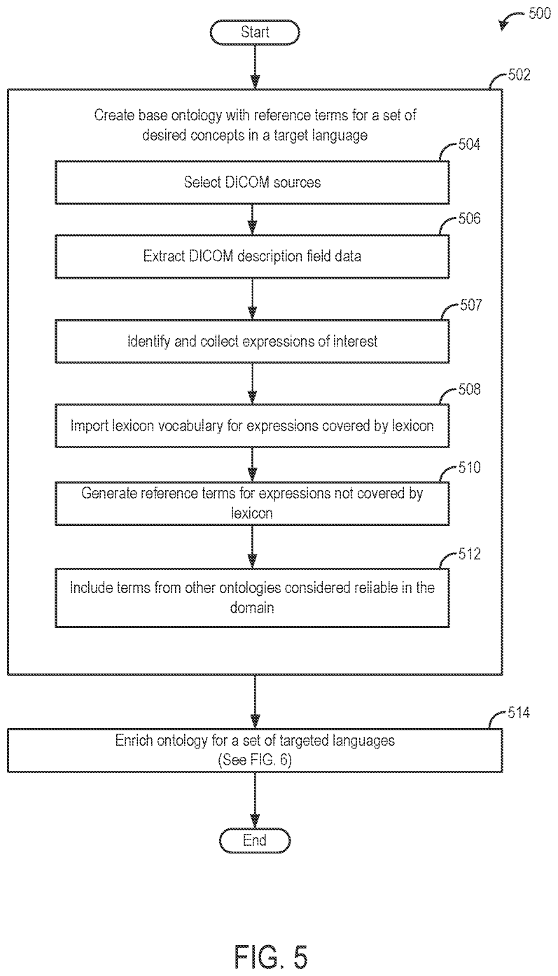

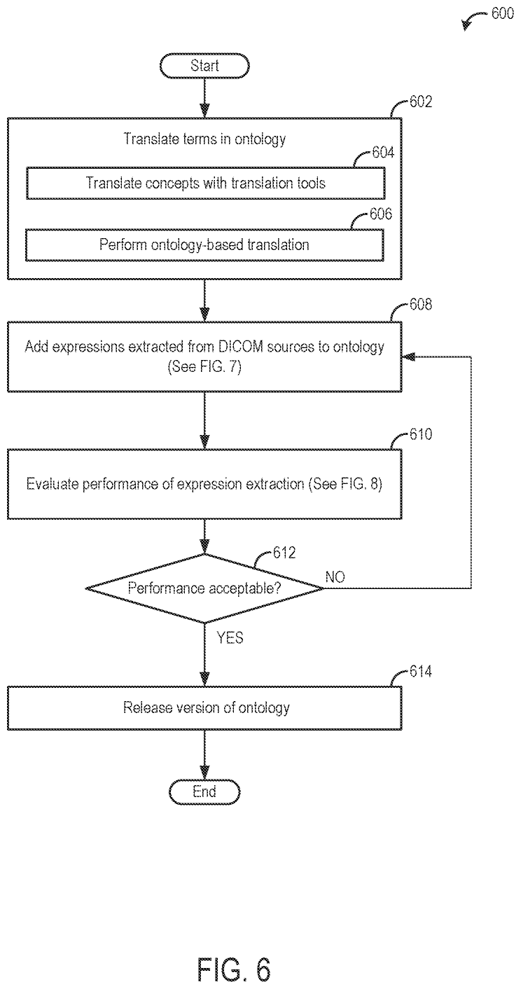

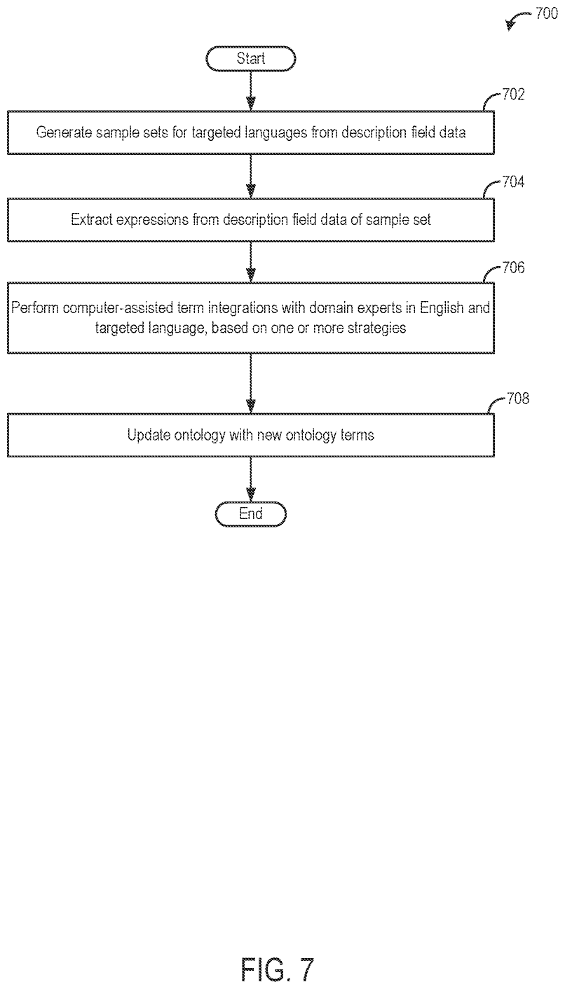

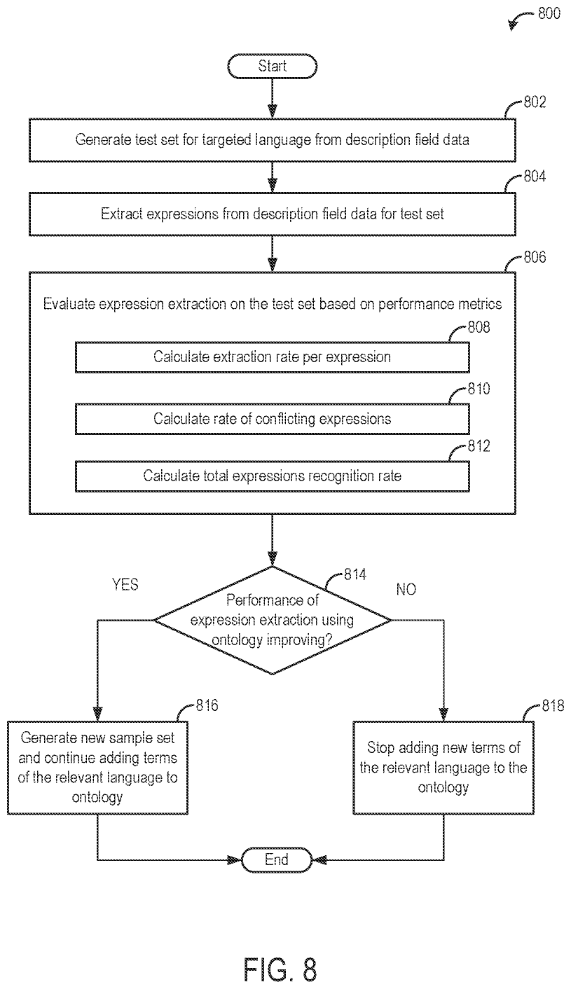

FIG. 1 shows a medical image processing system, where an automated workflow assistance service may be used to automatically select a medical exam reviewing application to use to review a medical exam. The automated workflow assistance service may intervene in a workflow of an image processing system, as illustrated in FIG. 2. The automated workflow assistance service may take as input a number of DICOM description fields, such as the exemplary DICOM description fields shown in FIG. 3A. The automated workflow assistance service may extract a plurality of expressions from the DICOM description fields, such as the expressions shown in FIG. 3B. The plurality of expressions may be matched to one or more lexicon entries, such as the exemplary lexicon entries shown in FIG. 3C. The medical exam reviewing application may be selected by following one or more steps of a method shown in FIGS. 4 and 5, based on an ontology. The ontology may be generated by following one or more steps of a method shown in FIG. 5, and enriched by following one or more steps of a method shown in FIG. 6. Enriching the ontology may be performed by following one or more steps of a method shown in FIG. 7. A performance of the ontology at recognizing and extracting expressions from the DICOM description fields may be evaluated by following one or more steps of a method shown in FIG. 8.

Referring to FIG. 1, an image processing system 100 is shown that provides automated workflow assistance. Image processing system 100 comprises a medical image processing device 102 and a user 150. Medical image processing device 102 may be a computing device, such as a desktop computer (e.g., a PC or a workstation), a laptop, a tablet, or different kind of computing device. In some embodiments, medical image processing device 102 may be an image processing device dedicated to reviewing images from medical exams, such as an image review server, an image post-processing system, a Picture Archiving and Communication System (PACS) system, an acquisition console on an acquisition machine, or a cloud multi-tenant image review service.

A medical exam reviewed by medical image processing device 102 may be stored as a set of DICOM files 140 (also referred to herein as a DICOM 140). DICOM 140 may include raw image data 142 acquired during the medical exam. DICOM 140 may also include a plurality of description fields 144, where each description field 144 may include one or more expressions relevant to the medical exam, an acquisition protocol of the exam, a series of the exam and/or images of the exam. The expressions may be single words, or multi-word expressions. For example, the expressions may include an anatomy of a patient; contrast information, including a contrast phase and a contrast agent; an acquisition gating; a laterality of raw image data 142; a pathology detected in raw image data 142; reconstruction filters used; a multi-energy indication; a weighting and/or pulse sequence, in the case of magnetic resonance (MR) images; and/or other options selected during the medical exam. Example expressions are described further below in reference to FIGS. 3A-3C.

Medical image processing device 102 may include a processor 104, a memory 106, and a user interface (UI) 120. Processor 104 may control an operation of medical image processing device 102 in response to control signals received at UI 120 from user 150. UI 120 may include a display (e.g., screen or monitor) and/or other subsystems. In some embodiments, UI 120 may be integrated into medical image processing device 102, where a user may interact with, adjust, or select control elements in the UI 120 (e.g., buttons, knobs, touchscreen elements, etc.) to send one or more control signals to the processor 104 from UI 120. In other embodiments, UI 120 is not integrated into the medical image processing device 102, and the user may interact with, adjust, or select control elements in UI 120 via a user input device, such as a mouse, track ball, touchpad, etc., or the operator may interact with UI 120 via a separate touchscreen, where the operator touches a display screen of UI 120 to interact with UI 120, or via another type of input device.

Processor 104 may execute instructions stored on the memory 106 to control medical image processing device 102. As discussed herein, memory 106 may include any non-transitory computer readable medium in which programming instructions are stored. For the purposes of this disclosure, the term “tangible computer readable medium” is expressly defined to include any type of computer readable storage. The example methods and systems may be implemented using coded instruction (e.g., computer readable instructions) stored on a non-transitory computer readable medium such as a flash memory, a read-only memory (ROM), a random-access memory (RAM), a cache, or any other storage media in which information is stored for any duration (e.g. for extended period time periods, permanently, brief instances, for temporarily buffering, and/or for caching of the information). In some embodiments, the non-transitory computer readable medium may be distributed across various computers and/or servers (e.g., provided via web services). Computer memory of computer readable storage mediums as referenced herein may include volatile and non-volatile or removable and non-removable media for a storage of electronic-formatted information such as computer readable program instructions or modules of computer readable program instructions, data, etc. that may be stand-alone or as part of a computing device. Examples of computer memory may include any other medium which can be used to store the desired electronic format of information and which can be accessed by the processor or processors or at least a portion of a computing device. In various embodiments, memory 106 may include an SD memory card, an internal and/or external hard disk, USB memory device, or similar modular memory.

Program code embodied on a computer readable medium may be transmitted using any appropriate medium, including but not limited to wireless, wireline, optical fiber cable, RF, etc., or any suitable combination of the foregoing. Computer program code for carrying out operations for aspects of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Various medical exam reviewing applications 110 may be installed on medical image processing device 102 for viewing, reviewing, navigating through, and/or analyzing images of a medical exam. Each medical exam reviewing application 110 may be suitable and/or preferred for a different type of medical exam. For example, a first medical exam reviewing application 110 may be used to view raw image data of a first type of medical exam; a second medical exam reviewing application 110 may be used to view raw image data of a second type of medical exam; a third medical exam reviewing application 110 may be used to view raw image data of a third type of medical exam; and so on.

Medical image processing device 102 may include an automated workflow assistance service 130, which may provide automated, AI-based assistance in selecting a suitable medical exam reviewing application 110 to launch to review a given medical exam, as described in greater detail in FIG. 2.

Referring now to FIG. 2, a data flow diagram 200 shows a flow of data through image processing system 100, indicated by a series of dashed lines. In various embodiments, reviewing a medical exam with a medical exam reviewing application 110 may include reviewing raw image data (e.g., radiology images) in accordance with a workflow 202. For the purposes of this disclosure, a workflow refers to a series of ordered steps carried out by a user on one or more medical images during a medical exam review task. For example, a workflow may include a first workflow step where a medical exam is loaded to review; a second workflow step where an application is launched to open to review the medical exam; a third workflow step where a desired layout of the application is selected; a fourth workflow step where a desired algorithm is selected to apply to data of the medical exam; a fifth workflow step where the user performs an analysis of the first image using a tool; and so on. One or more decision points may occur at various points in a workflow, where a decision point is a point in the workflow where a subsequent workflow step depends on a decision made by a user (e.g., user 150).

Workflow 202 may be an initial portion of a workflow, where, in a first file load step 204, a set of DICOM files 140 of a medical exam to review is loaded. For example, user 150 may be prompted to select a medical exam to load. Workflow 202 may then proceed to an application selection step 206, where a suitable medical exam reviewing application 110 may be selected to review the loaded medical exam. At application selection step 206, automated workflow assistance service 130 may provide assistance with selecting a suitable medical exam reviewing application 110. After the suitable medical exam reviewing application 110 has been selected, workflow 202 may proceed to an application launch step 208, where the selected medical exam reviewing application 110 may be opened. After the selected medical exam reviewing application 110 is opened, user 150 may proceed to review the loaded medical exam in the selected medical exam reviewing application 110.

To select the suitable medical exam reviewing application 110, automated workflow assistance service 130 may use an ontology 220 to match expressions extracted from description fields of the DICOM file 140 to entries in a corresponding radiology lexicon, such as the RadLex lexicon. As described in greater detail below, the matching lexicon entries may be used to select the suitable medical exam reviewing application 110. FIG. 3A shows example description fields used as input, and a set of expressions extracted from the description fields using ontology 220 is shown in FIG. 3B. An example set of radiology lexicon entries matching the extracted set of expressions is shown in FIG. 3C. FIGS. 3A, 3B, and 3C are described below in reference to FIG. 4A.

Referring now to FIG. 4A, an exemplary method 400 is shown for generating a prioritized list of lexicon entries that match expressions extracted from description fields of a set of DICOM files of a medical imaging exam. Method 400 and other methods described herein are described with reference to a medical image processing system, such as image processing system 100 of FIG. 1, and in particular, an automated workflow assistance service, such as automated assistance service 130. Method 400 and the other methods described herein may be implemented via computer-readable instructions stored in a memory of a medical image processing device, and executed by a processor of the medical image processing device, such as memory 106 and processor 104 of medical image processing device 102 of FIG. 1.

Method 400 begins at 402, where method 400 includes extracting expressions (e.g., text descriptions) from description fields of a DICOM file of the medical exam. In various embodiments, the medical exam is loaded onto the medical image processing device as a set of DICOM files by a user of the medical image processing device. The description fields may include information about why and how raw image data of the medical exam was acquired. An example of expressions included in the DICOM file is shown in FIG. 3A.

Turning to FIG. 3A, a set of example description fields 300 of a DICOM file is shown. The set of example description fields 300 includes a modality description field 302, which may include a standardized description of a modality of a medical imaging system used to acquire raw image data (e.g., raw image data 142 of FIG. 1). For example, the standardized description included in modality description field 302 indicates that the modality was magnetic resonance (MR). For other DICOM files, the modality may be different. The modality information may be defined at a series level, meaning files (e.g., images with associated DICOM headers) belonging to a same series may have the same modality. In other examples, the modality may be computed tomography (CAT), ultrasound, positron emission tomography (PET), mammography, fluoroscopy, bone density (DEXA), X-Ray, or a different type of imaging modality.

The description fields 304, 305, 306 described herein may include an expression that is generated automatically. For example, the expression may be generated by a software application of an image processing system used to acquire the raw image data when the DICOM file is created. Alternatively, the description fields described herein may include expressions that are generated manually, where a user (e.g., a radiologist) enters a textual description using an input device of the image processing system or a different computing device. For example, during creation of the DICOM file, the user may be prompted to enter in the textual description, or after creation of the DICOM file, the user may choose to enter additional textual information into one or more description fields of the DICOM file. It should be appreciated that description fields, as described herein, refer to any field including non-standardized text where processing is used to extract a relevant expression, and not simply fields with a “description” label. Additionally, while DICOM description fields are described herein, in other embodiments, expressions may be extracted from description fields of other types of files including patient data.

The set of example description fields 300 includes a study description field 304, which may include a textual description of a study performed during acquisition of the raw image data. For example, study description field 304 indicates that a study of an ankle of a patient was performed. The textual description included in study description field 304, as well as the descriptions of other description fields, may be in a different language than a language of a radiologist, and/or a different language than other description fields of the DICOM file. In FIG. 3A, the study description is “rm tobillo”, where the field is described in the Spanish language (e.g., “resonancia magnetica”). In other cases, the second, study description may be in French, or German, or a different language. Further, the study description may include portions in a first language, and portions in a second language.

The set of example description fields 300 includes a protocol name description field 306, which may include a textual description indicating a name of a protocol used during acquisition of the raw image data. For example, protocol name description field 306 indicates an expression tobillo./3, indicating a third ankle protocol.

The set of example description fields 300 includes a series description field 308, which may include a textual description of a series of images of the raw image data. For example, series description field 308 includes the textual description “sag gre t2”, indicating that a corresponding series images of raw image data are acquired at a sagittal acquisition plane and a gradient echo pulse sequence.

The DICOM file may include a plurality of some types of description field. For example, the DICOM file may include a plurality of protocol name fields, and/or a plurality of series description fields. For example, a study described in a study description field may include a plurality of protocols, each protocol of which may be described in a protocol name field. Each protocol of the plurality of protocols may include a plurality of series of images, each series of which may be described in a series description field.

Returning to method 400, at 404, method 400 includes pre-processing the expressions extracted from the DICOM description fields. In various embodiments, the expressions may be pre-processed by the automated workflow assistance service to normalize and/or regularize the expressions into a standard format, where the standard format matches elements of a pre-established ontology (e.g., ontology 220 of FIG. 2). The ontology may be used for mapping the expressions to entries of a lexicon. In some embodiments, the pre-processing of an expression may include a plurality of stages, during which different types of pre-processing are carried out on the expression.

Pre-processing of the expressions may also include a tokenization of text in a description field, where the text is segmented into words. For example, a text description may include a plurality of words that may be combined into a single expression (e.g., magnetic resonance), and the text description may include words that represent individual expressions (e.g., ankle). Thus, the pre-processing may resolve a plurality of words of the text descriptions into one or more suitable single- or multi-word expressions. It should be appreciated that the examples included herein are for illustrative purposes, and other types of pre-processing may be carried out on the text descriptions without departing from the scope of this disclosure.

At 406, method 400 includes identifying one or more languages of the expressions extracted from the DICOM description fields. For example, a language of the expression may be identified, by looking up the expression in different reference databases in different languages. Identifying the language makes it possible to specialize a conversion of a plurality of words into an expression, per language, by taking into account a syntax of the language. It may also increase a computational speed with which the ontology is used during deployment by limiting the number of possible options.

At 408, method 400 includes mapping expressions extracted from the text descriptions of the DICOM description fields to corresponding concepts of an ontology (e.g., ontology 220). An ontological concept may be defined by a term of the ontology that matches the expression, as well as a path through nodes of the ontology to arrive at the term. The expressions may be compared to a plurality of terms in the ontology, to determine a closest matching term.

Turning to FIG. 3B, an exemplary set of concepts 330 of an ontology is shown, where the concepts 330 match expressions extracted from the description fields of FIG. 3A. Each concept is represented in FIG. 3B as a path leading from a top level concept of the ontology down to a term included in the ontology under the concept. Some concepts may include one or more categories, or hierarchical levels of categories, between the top level concept and the term.

For example, exemplary set of concepts 330 includes a first concept 332 “Modality|MR”, which matches the expression “MR” extracted from modality description field 302 indicating that the raw image data was acquired via magnetic resonance. In the ontology, “Modality” is a top level concept, and “MR” is a term hierarchically positioned under “Modality”. For the first concept 332, no processing may have been carried out, where the extracted expression “MR” of the modality description field 302 may be a close or exact match with a corresponding term/path of the ontology.

The exemplary set of concepts 330 includes a second concept 334 “Anatomy ankle”, which matches the expression “rm tobillo” extracted from study description field 304 (indicating that a study was performed on an anatomical region of an ankle of a patient). In the ontology, “Anatomy” is a top level concept, and “ankle” is a term hierarchically positioned under “Anatomy”. In other words, the Spanish word “tobillo” included in the study description field 304 of FIG. 3A may be resolved into English (e.g., a reference language used by the ontology) by the automated assistance service in a first step, and the corresponding English word “ankle” may be mapped to the ontological path “Anatomy|ankle” in a second step.

In some embodiments, a plurality of text descriptions of a DICOM file may resolve to a single term/path in the ontology. For example, a third concept 336 “Anatomy|ankle” may be extracted for a protocol name, where the expression extracted for the protocol name is the same expression extracted for the study description. Alternatively, a single text description of a DICOM file may resolve to a plurality of expressions in the ontology. For example, the text description included in series description field 308 of FIG. 3A, “sag gre t2”, is resolved in FIG. 3B into three distinct ontological concepts 338, 340, and 342. Concepts 338, 340, and 342 indicate that an orientation of the raw image data is on a sagittal acquisition plane, that one or more MRI sequences of the raw image data include a gradient echo pulse sequence, and that the one or more MRI sequences are of the type T2.

Expression extraction using the ontology may include additional text processing steps. For example, a negation present in a textual description may be addressed, or a proximity of terms in the textual description may be analyzed, or a different type of processing may be performed. One or more abbreviations of the textual description may be expanded, or the textual description may be converted to a desired abbreviation. Additionally, proximity measures may be used to match a word including a spelling mistake with an term included in the ontology.

Returning to FIG. 4A, at 410, method 400 includes combining the concepts of the ontology that match the extracted expressions across different levels, including the exam level, the protocol level, and the series level. In various embodiments, the concepts may be combined using a combination logic defined for each concept, based on a knowledge graph encoding the rules to be applied when performing the combination, and a reasoning engine to apply these rules.

At 412, method 400 includes mapping the combined ontological concepts to entries of a radiology lexicon. In other words, the combined ontological concepts may be matched with one or more entries of a radiology lexicon with a similar hierarchical structure, where the one or more entries are included at a similar hierarchical location of the radiology lexicon. A combination of ontological concepts generated at 410 may match a single lexicon entry to various degrees. Some concepts may have an exact match, while other concepts may roughly match more than one lexicon entry. Thus, for the combination of ontological concepts, a list of candidate lexicon entries may be generated.

At 414, method 400 includes prioritizing the list of lexicon entries based on the combined concepts. For example, the combined concepts may match most closely with a first lexicon entry, and may match less closely with a second lexicon entry and a third lexicon entry. In various embodiments, a degree of closeness of a match may be based on a specificity of the combined concepts. In other words, if the combined concepts are very specific (e.g., where the combined concepts result from a larger number of expressions extracted from DICOM description fields), the combined concepts may match closely with a lexicon entry. If the combined concepts are not very specific (e.g., where the combined concepts result from a smaller number of expressions extracted from DICOM description fields), the combined concepts may match less closely with various lexicon entries.

As an example of how method 400 may be used, a radiologist may wish to review an imaging exam described by a set of DICOM files. An exam level description field of a DICOM file of the set of DICOM files may include a pathology of the patient, and an anatomical area of the patient. The automated workflow assistance service may extract a first set of expressions from the exam level description field, where the first set of expressions may include the pathology, the anatomical area of the patient, and a name of a radiologist treating the patient. A second set of expressions may be extracted from a modality description field of the DICOM file, where the second set of expressions includes the modality. A third set of expressions may be extracted from one or more study description fields of the DICOM file, where the third set of expressions includes descriptions of one or more studies included in the DICOM file. A fourth set of expressions may be extracted from one or more protocol name fields of the DICOM file, where the fourth set of expressions includes descriptions of one or more protocols included in each study of the one or more studies. For example, a single study may include 3 protocols. A fifth set of expressions may be extracted from one or more series description fields of the DICOM file, where the fifth set of expressions includes descriptions of one or more series of images included under each protocol of the one or more protocols. For example, a single protocol may include several dozen series of images. To be successfully launched, a given application may rely on a minimum set of series types.

The automated workflow assistance service may consult the ontology to determine which concepts of the ontology most closely match each extracted expression. A list of matching ontological concepts may then be extracted from the ontology, where each matching ontological concept includes path and node information of the ontological concept within the ontology. The list of matching ontological concepts may then be combined, using combination logic where a series of rules are applied based on a knowledge graph. The combination of ontological concepts (e.g., a combined ontological concept) may then be mapped to one or more entries of a chosen radiology lexicon sharing a similar hierarchical structure as the ontology.

In other words, a correspondence may be established between the expressions extracted from the exam, and matching reference terms of the ontology. A correspondence may subsequently be established between the matching reference terms of the ontology and categories and terms used in the lexicon. A list of matching lexicon entries may be generated and prioritized to determine one or more closest matches to the original expressions and values extracted from the DICOM files.

Turning to FIG. 3C, an exemplary set of lexicon entries 360 is shown, where exemplary set of lexicon entries 360 is based on exemplary set of concepts 330 of FIG. 3B, extracted from the description fields of FIG. 3A. Exemplary set of lexicon entries 360 includes a first lexicon entry 362, which represents a closest match in the ontology to a combination of the expressions of the exemplary set of concepts 330. Thus, first lexicon entry 362 is a most probable and specific lexicon entry of exemplary set of lexicon entries 360. Exemplary set of lexicon entries 360 also includes two additional lexicon entries, which match the combination of the expressions of the exemplary set of concepts 330 less closely and/or with lower specificity. A second lexicon entry 364 represents a next closest match in the ontology to the combination of the expressions of the exemplary set of concepts 330, and a third lexicon entry 366 represents a least closest/specific match in the ontology to the combination of the expressions of the exemplary set of concepts 330. In other words, first lexicon entry 362 “MR Ankle” is more specific than second lexicon entry 364 “MR Lower extremity”, which in turn is more specific than third lexicon entry 366 “MR unspecified body region”.

As an alternative approach, for each expression, a reference term in our ontology and/or a category in the lexicon for a expression may be more or less specific. For example, liver is more specific than chest, as the liver is an organs located in the chest. This level of specificity of a reference term/category within one expression can be assigned a weight. Then, each expression can have a weight, to represent the importance of the expression for the matching entries of the lexicon. For example, the expressions “Anatomy” and “Contrast” may be assigned higher weight coefficients, while a “Laterality” may be assigned a lower weight coefficient. Both types of weights may be combined in various ways (e.g., multiplied, added, etc.).

At 416, method 400 includes storing the prioritized list of lexicon entries in a memory of the medical image processing device (e.g., memory 106), where the prioritized list may be accessed by the automated workflow assistance service during one or more steps of a workflow of the radiologist while the radiologist is reviewing the medical imaging exam. Method 400 ends.

Thus, by performing method 400, expressions included in a set of DICOM fields of a medical imaging exam may be mapped, using the ontology, to a prioritized list of entries of a radiology lexicon, such as the RadLex lexicon. Once the prioritized list of entries matching the expressions has been generated, the prioritized list of entries may be used to aid a user of the medical image processing device in navigating various options for configuring the medical image processing device for reviewing the medical imaging exam. Use of the prioritized list of lexicon entries is described in greater detail below in reference to FIG. 4B.

Referring now to FIG. 4B, an exemplary method 450 is shown for an automated workflow assistance service of a medical image processing device, for using a prioritized list of lexicon entries associated with a medical imaging exam to automate one or more steps of a workflow of a user of the medical image processing device (e.g., when reviewing the exam). The prioritized list of lexicon entries may be based on expressions extracted from description fields of a set of DICOM files of the medical exam, as described above in reference to FIG. 4A. The automated workflow assistance service may be a non-limiting example of automated assistance service 130 of FIG. 1.

Method 450 begins at 452, where method 450 includes receiving the prioritized list of lexicon entries. The prioritized list of lexicon entries may be accessed from a memory of the medical image processing device (e.g., memory 106) by the automated workflow assistance service, where the prioritized list was stored as described above in reference to FIG. 4A. As described above, the prioritized list of lexicon entries may be used by the automated workflow assistance service to aid a user of the medical image processing device in reviewing the medical imaging exam. Specifically, the prioritized list of entries may be used at various decision points in a workflow of the user, to automatically select or help the user select a suitable option, based on the decision point. For example, at one decision point, the user may be prompted to launch a suitable application for reviewing a portion of the medical imaging exam. At a different decision point, the user may be prompted to select a layout of the suitable application, or an algorithm to apply to data of the medical exam, or a configuration of the suitable application and/or medical imaging processing device.

At 454, method 450 includes receiving a list of available options pertaining to a current step of the workflow. In other words, the list of available options may be a set of options for configuring software of the medical image processing device to review or continue to review the medical imaging exam. For example, if the current step of the workflow includes selecting an appropriate application to review the medical exam, the list of available options may be a list of candidate applications to launch, where one candidate application of the list of candidate applications may be a most suitable application for reviewing the medical imaging exam.

At 456, method 450 includes determining whether a matching lexicon entry of the prioritized list of lexicon entries (e.g., a most probable and specific lexicon entry) is identified. In various embodiments, the matching lexicon entry may be a lexicon entry that maps to one option of the list of available options, where other lexicon entries do not map to options of the list of available options. If at 456 it is determined that a matching lexicon entry is identified, method 450 proceeds to 458. At 458, method 450 includes selecting a most suitable option based on the matching lexicon entry. For example, the most suitable option may be an application to launch to review the portion of the medical imaging exam, or a layout option of the application, or a different configuration option. At 460, method 450 includes implementing the selected most suitable option (e.g., launching a selected application, loading a selected layout option, etc.), and method 450 ends.

Alternatively, if at 456 it is determined that a matching lexicon entry is not identified, method 450 proceeds to 462. At 462, method 450 includes prompting the user to select a desired option from the list of available options. In various embodiments, the list of available options is displayed as menu options on a display screen of the medical image processing device, where the user may select one or more options of the menu options. At 464, method 450 includes receiving a selected option from the user, and method 450 proceeds to 460, where the selected option is implemented, and method 450 ends.

In some embodiments, a number of lexicon entries on the prioritized list of lexicon entries may be reduced. For example, lexicon entries from the prioritized list of lexicon entries that are below a threshold relevance may be eliminated, where the threshold relevance is determined based on how close a lexicon entry matches an original set of extracted expressions. By reducing the number of lexicon entries on the prioritized list of lexicon entries, a number of options displayed to the user may be reduced, where options that have a low probability of being selected by the user are not displayed.

Thus, the prioritized list of lexicon entries serves to characterize and describe the medical imaging exam in a semi-standardized manner, such that one or more lexicon entries distinguishes a type of the medical imaging exam with sufficient accuracy and precision that the one or more lexicon entries can be used to make automated decisions with respect to configuration and task-related options that arise during an exam review workflow. If the one or more lexicon entries closely match one of the options, the matching option may be automatically selected and implemented. If a close match is not found, a short list of the next closest lexicon entries may be presented to the user for selection, where the short list includes a smaller number of options than the list of available options.

Referring now to FIG. 5, an exemplary method 500 is shown for creating an ontology which may be used to match expressions extracted from description fields of a DICOM file with corresponding lexicon entries, as described above in reference to FIGS. 4A and 4B. The ontology may be a non-limiting example of ontology 220 of FIG. 2, and may be used by an automated assistance service, such as automated assistance service 130 of FIGS. 1 and 2. In various embodiments, the ontology may be first generated in a base language and then expanded to include other languages. In various embodiments, the base language is English.

Method 500 begins at 502, where method 500 includes creating a base ontology with reference terms corresponding to a set of desired, ontologically linked concepts in a first, reference language. The set of desired concepts may cover expressions likely to be included in one or more description fields of a DICOM file of a medical imaging exam. The reference terms may be organized as ontological concepts defined by a path through the ontology, including a starting or top level node of the ontology and ending at a reference term of the ontology that matches an expression of the set of desired expressions. The one or more description fields may relate to the medical imaging exam, or to a protocol used to acquire images in this exam, or more specifically to a series or group of images acquired during the medical imaging exam. In various embodiments, the base ontology may be created via a procedure that combines automated software routines with manual curation techniques and steps. These procedures may use knowledge graphs and inferencing techniques.

At 504, creating the base ontology includes selecting a plurality of DICOM sources. The DICOM sources may be DICOM files generated during previous or historical medical imaging exams. The DICOM files may be stored in one or more databases accessible to one or more hospital networks. In some embodiments, the DICOM files may be anonymized DICOM files available in public databases. In various embodiments, desired DICOM sources (e.g., of a plurality of possible DICOM sources) may be defined in a first step, and the information from the DICOM files may be obtained in a second step.

At 506, creating the base ontology includes extracting DICOM description field data from the DICOM sources. The description field data may be retrieved programmatically from the DICOM sources. The description field data may be retrieved in more than one way. Some of the description field data may be indexed by a Picture Archiving and Communications System (PACS), where the description field data may be retrieved programmatically from the PACS system. In other cases or embodiments, the description field data may be retrieved from log files generated when imaging exams are performed. For example, a log file may indicate that one or more text expressions and/or words in the log file are taken from a relevant description field of a DICOM file. When the description field data is retrieved, any personal data pertaining to a patient and/or to a healthcare professional may be removed to anonymize the description field data.

At 507, creating the base ontology includes identifying expressions of interest from the description fields of the DICOM sources. Each description field may have a header that identifies a type of expression included in the description field. For example, a header of a description field may be “Anatomy”, where the description field may include an expression corresponding to an anatomical region, such as “Ankle”. Other examples of headers found in DICOM description fields include laterality, pathology, contrast, contrast phase, contrast agent, acquisition gating, reconstruction filters, and/or multi-energy. Some headers may be specific to certain types of image acquisition. For example, for magnetic resonance imaging, headings may include weighting and/or pulse sequence. It should be appreciated that the examples provided herein are for illustrative purposes, and additional and/or different expressions may be included without departing from the scope of this disclosure.

To identify which expressions are worth covering in the ontology, two approaches may be taken, which may be combined. In a first approach, a sample of real-world description fields may be examined to see what type of expressions are present and would be desirable to capture. In a second approach, a target lexicon to which we wish to map the exam, protocol, or series may be examined, to see what expressions are used. Certain lexicons such as RadLex may be built on a specific set of expressions (e.g., each RadLex protocol lexicon entry is defined by a plurality of values corresponding to a respective plurality of expressions).

At 508, creating the base ontology includes importing lexicon vocabulary for expressions covered by the lexicon. The lexicon vocabulary may include categories (or additional reference terms) for each expression from one or more radiology lexicons, for expressions of interest covered by the one or more radiology lexicons. In various embodiments, the one or more radiology lexicons may include the RadLex lexicon. For example, the one or more radiology lexicons may be searched for an extracted expression. If the extracted expression is found, additional terms of the one or more radiology lexicons associated with the extracted expression may be imported as terms into the ontology. By importing the associated additional terms, a wider range of vocabulary related to the collected expression of interest may be identified and included in the ontology.

At 510, creating the base ontology includes generating a set of reference terms to include in the ontology for expressions not covered by the one or more lexicons. For various expressions identified and collected from the DICOM sources, no related lexicon vocabulary may be identified. When no related lexicon vocabulary is identified for an extracted expression, reference terms may be generated to further widen the range of vocabulary related to the extracted expression. In various embodiments, the reference terms may be defined manually by a human expert in a related domain. In other embodiments, the reference terms may be generated programmatically, for example, by consulting online reference sites and/or materials.

At 512, creating the base ontology includes including terms from other ontologies considered reliable in the domain. For example, a reference term included in the ontology (or imported from a lexicon, or reference database) may also be included in one or more different ontologies. A software program may search the one or more different ontologies for vocabulary including or related to the term. If a relevant expression is discovered, the relevant vocabulary may be imported into the ontology and associated with the term in the ontology. This process may be controlled by an expert to ensure a given expression is not covered by multiple reference terms.

At 514, method 500 includes enriching the ontology with other expressions likely to be found in DICOM description fields, and content in other languages, for a set of targeted languages. For example, a reference term of the ontology may be translated from a language of the ontology into a second language. One or more radiology lexicons, ontologies, or other reference materials in the second language may be searched for vocabulary including or related to the translated reference term. If one or more expressions are found, the one or more expressions may be associated with the reference term in the ontology. In this way, a set of lexicon entries of a first language may be associated with terms of other languages included in the ontology. Enriching the ontology with content in the other languages is described in greater detail below in reference to FIG. 6.

Referring now to FIG. 6, an exemplary method 600 is shown for enriching a base ontology, in a given language, by integrating additional reference terms that correspond to ontological concepts of the ontology. The additional reference terms may be derived from expressions extracted from description fields of DICOM files, as described above, or other sources. The other sources may include sources in different languages. The ontology may be a non-limiting example of ontology 220 of FIG. 2, and may be used by an automated assistance service, such as automated assistance service 130 of FIGS. 1 and 2, running on a computing device of a medical imaging system (e.g., image processing system 100).

Method 600 begins at 602, where method 600 includes translating the reference terms and ontological concepts of the ontology into a set of target languages. For example, the set of targeted languages may be a set of languages supported by the medical imaging system or medical exam reviewing software running on the computing device.

The translation may be performed using various methods. At 604, translating the expressions includes using translation tools. For example, reference terms and/or concepts included in the ontology in the reference language may be inserted as inputs into translation software, where translated expressions in a target language may be an output of the translation software. The translated expressions may be added to the ontology at a hierarchical location corresponding to corresponding terms in the reference language. In some embodiments, more than one type of translation software may be used.

At 606, translating the expressions may include using ontology-based translation methods. For example, the ontology may be compared to a second ontology including anatomical terms in a second language. By exploiting similarities between the ontology and the second ontology, foreign language terms from the second ontology may be imported into hierarchically corresponding areas of the ontology. For example, the base ontology may be an English ontology. A French ontology may include a similar hierarchical structure as the English ontology, whereby terms included in French in the French ontology may be imported into the English ontology as synonyms to English terms in corresponding sections of the English ontology.

At 608, method 600 includes adding expressions extracted from DICOM sources, including foreign language DICOM sources, to the ontology. To some extent, the reference terms in the ontology may differ from terms used in practice by radiologists. For example, a reference term of the ontology may be a long-form translation of an extracted expression, where a short-form or abbreviated term may be more commonly used in practice by radiologists. In each targeted language, radiologists may use different types of abbreviations. Thus, to ensure that a comprehensive set of terms are included in the ontology, an ontology enrichment procedure may be performed to widen a breadth of terms included the ontology to cover a range of terms used in practice. An exemplary procedure is described in greater detail below in reference to FIG. 7.

At 610, method 600 includes evaluating a performance of the expression extraction carried out at 608. An exemplary procedure for evaluating the performance is described in greater detail below in reference to FIG. 8.

At 612, method 600 includes determining whether the performance of the expression extraction across the targeted languages is considered acceptable. If the performance of the foreign language expression extraction is not considered acceptable, method 600 proceeds back to 608, where the ontology enrichment procedure may be repeated. If the performance of the expression extraction is considered acceptable, method 600 proceeds to 614.

At 614, method 600 includes releasing a version of the ontology. Once released, the ontology may be used by the automated assistance service in aiding a radiologist in navigating various workflow options while reviewing an exam, as described above in reference to FIGS. 4A and 4B.

Referring now to FIG. 7, an exemplary method 700 is shown for enriching an ontology with expressions extracted from DICOM sources, including foreign language DICOM sources, to ensure a coverage of actual language usage in one or more targeted languages. The ontology may be a non-limiting example of ontology 220 of FIG. 2, and may be used by an automated assistance service, such as automated assistance service 130 of FIGS. 1 and 2.

Method 700 begins at 702, where method 700 includes generating sample sets for the one or more targeted languages from description field data of one or more DICOM sources (e.g., the DICOM files 140 of FIG. 1) in the targeted languages. In various embodiments, separate sample sets may be created for different targeted languages. For example, a first sample set may be generated by extracting description field data of one or more DICOM sources in the English (e.g., reference) language; a second sample set may be generated by extracting description field data of one or more DICOM sources in the French language; a third sample set may be generated by extracting description field data of one or more DICOM sources in the German language; and so on.

Generating the sample set may include collecting DICOM description field data from various sources, as described above in reference to method 500 of FIG. 5. For example, the description field data may be retrieved programmatically from a PACS system of a relevant language, or from log files generated in the relevant language when imaging exams are performed, or from one or more DICOM files created from exams performed by radiologists in the relevant language, or in a different manner.

In various embodiments, method 700 may be performed a plurality of times in an iterative manner, for each target language. For example, a first set of additional reference terms may be added to the ontology based on the first sample set; a second set of additional reference terms may be added to the ontology based on the second sample set; and so on. With each subsequent sample set, the terms of the ontology may more accurately cover a wide range of expressions used in practice by radiologists in various languages.

As the sample sets may be drawn from a same or similar sets of DICOM description field data, a measured improvement of the ontology over subsequent sample sets may indicate an increased coverage of the ontology, until the coverage of the ontology is determined to be sufficient on each targeted language. As used herein, coverage may refer to a comprehensiveness of a set of terms of the ontology with respect to covering instances of various expressions or expressions found in the DICOM description field data. Measuring the performance of the ontology is described in greater detail below in reference to FIG. 8.

At 704, method 700 includes identifying and extracting expressions from the description field data, as described above in reference to method 500. Extracting the expressions from the description field data may include processing the description field data. For example, references to a patient or radiologist name or identifying information of the patient may be removed, to anonymize the description field data and protect a privacy of the patient and of the radiologist. Extraneous words may also be removed, such as, for example, when a radiologist enters text in a description field in a sentence or other natural language format. Numeric values may be identified and, depending on the local language syntax, preceding and or succeeding words may be extracted to capture the nature of the numeric information (e.g. weight) and the unit of the numeric information (e.g. kg).

At 706, method 700 includes performing computer-assisted term integrations into the ontology using domain experts in both English and in the targeted language. During the computer-assisted term integrations, various strategies may be used to ensure an accuracy and a consistency of the expressions imported into the ontology from the targeted languages.

A first strategy of the various strategies may include reviewing translated extracted expressions for accuracy in translation. In some cases, translation errors may be discovered, which may be corrected by a domain expert. A second strategy may include measuring a frequency of expressions extracted from the DICOM sources that are not recognized in the ontology. Expressions extracted from the DICOM sources that are not recognized in the ontology (e.g., that do not match any terms of the ontology) may be collected, and then ordered by a frequency with which the expressions are found in the DICOM sources. A threshold frequency may be selected, and all expressions with a frequency above the threshold frequency may be manually selected for adding to the ontology by the domain expert, if not found within the ontology.

As another strategy, conflicting expressions within the DICOM sources may be identified and reviewed by the domain expert. For example, a protocol name may be extracted from the DICOM sources. A first expression may be extracted from the DICOM sources in relation to the protocol name that includes a first anatomical region of a patient. A second expression that matches the first expression may be extracted from the DICOM sources in relation to the protocol name, which includes a second anatomical region of a patient. A probability of the protocol name being applicable to both the first anatomical region and the second anatomical region may be below a threshold probability (e.g., 2%). As a result of the probability being below the threshold probability, a computer program may review the extracted expressions, and may flag the first expression as potentially conflicting with the second expression, whereby the first expression and the second expression may be presented to the domain expert to be reconciled. For example, in one embodiment, the first expression and the second expression may be displayed concurrently in a UI of the computer program. The domain expert may determine that either or both of the first expression or the second expression are invalid (e.g., due to a mistake made by a radiologist in entering text into a description field of a DICOM file or because of an error in the ontology), whereby the invalid expression may not be selected to be included in the ontology. Alternatively, the domain expert may determine that both of the first expression and the second expression are valid, whereby the first expression and the second expression may both be selected to be included in the ontology.

At 708, method 700 includes updating the ontology with the new ontology terms identified in steps 702-706. In some embodiments, the ontology may be updated via an automated procedure during or after the computer-assisted term integration procedure. For example, some expressions extracted from the DICOM sources may be automatically added to the ontology, based on a logic of the automated procedure. In other cases, the updating of the ontology may be performed manually by the domain expert. In some embodiments, the manual updating may be performed during the computer-assisted term integration procedure, on a case-by-case basis. In other embodiments, the manual updating may be performed after the computer-assisted term integration procedure has ended. For example, during the computer-assisted term integration procedure, the domain expert may generate a list of changes to be made to the ontology, and after the computer-assisted term integration procedure, the changes on the list may be applied to the ontology by a second automated procedure. Further, in some embodiments, updating of the ontology may be performed under the supervision of an ontology administrator.

It should be appreciated that the strategies included herein for reviewing the terms of the ontology are for illustrative purposes, and in other embodiments, other strategies may be used without departing from the scope of this disclosure. Additionally, the computer-assisted term integration procedure may be flexible, where the domain expert may be free to delete, add, or change terms of the ontology. For example, the domain expert may add a synonym of a term to the ontology, or modify an attachment of a term to a reference term of the ontology.

Referring now to FIG. 8, an exemplary method 800 is shown for evaluating a performance of the expression extraction carried out via method 700 of FIG. 7. Method 800 begins at 802, where method 800 includes generating a test set for a targeted language from description field data of one or more DICOM sources (e.g., the DICOM files 140 of FIG. 1), as described above in reference to method 700. In various embodiments, separate test sets may be created for various targeted languages. For example, the base ontology may be in the English language; a first test set may be generated by extracting description field data of one or more DICOM sources in the French language; a second test set may be generated by extracting description field data of one or more DICOM sources in the German language; and so on.

At 804, method 800 includes identifying and extracting expressions from the description field data. In various embodiments, the test set may be generated concurrently and/or via a similar process as used in the generation of the sample set, as described in reference to method 700 above. For example, a set of extracted description field data may be generated, and the set of extracted description field data may subsequently be divided into the sample set and the test set. The expressions may also be extracted from the set of extracted description field data, and the extracted expressions may subsequently be divided into the sample set and the test set. In various embodiments, the sample set may be larger than the test set.

At 806, method 800 includes evaluating expression extraction on test sets generated in the different targeted languages using the ontology, based on one or more performance metrics. In various embodiments, the expression extraction may be evaluated by comparing measurements of the performance metrics with previous measurements made with previous versions of the ontology. For example, as described above, the ontology may be enriched with new reference terms via an iterative procedure, where a series of sample sets are used to iteratively update and refine terms of the ontology until the terms of the ontology match expressions extracted from an expansive body of DICOM description fields above a threshold degree of closeness or correlation.

At 808, evaluating the expression extraction may include calculating an extraction rate per expression, over various iterations in each targeted language. During each iteration of the ontology, for each expression extracted from the test set, a number of instances of the expression extracted from the DICOM description fields may be counted. For example, after a first iteration of ontology enrichment, a first number of instances of the expression may be extracted from a test set. After a second iteration of ontology enrichment (e.g., with a new or expanded sample set), a second number of instances of the expression may be extracted from the test set. After a third iteration of ontology enrichment, a third number of instances of the expression may be extracted from the test set. By comparing the number of instances extracted over the various iterations, the extraction rate per expression may be determined.

For example, if 10 instances of an expression are extracted after each of the first iteration of ontology enrichment, the second iteration of ontology enrichment, and the third iteration of ontology enrichment, the extraction rate per expression may be unchanged at 10 instances per iteration. If the extraction rate per expression is unchanged, subsequent iterations of method 700 with additional sample sets may be performed. Alternatively, if 10 instances of an expression are extracted after the first iteration, 15 instances are extracted after the second iteration, and 20 instances are extracted after the third iteration, the extraction rate per expression may be increasing. An increasing extraction rate per expression may be an indication that a coverage of the ontology for the relevant language is approaching a sufficient coverage. If, after various iterations of method 700 have been performed, the extraction rate per expression remains stable and high, it may be inferred that sufficient coverage has been achieved.

At 810, evaluating the expression extraction may include calculating a rate of conflicting terms per expression, over various iterations of ontology enrichment in each targeted language. Identification of conflicting terms may be based on a comprehensiveness of the ontology, where an ontology with a greater number of reference terms may identify more conflicting terms in description field data than an ontology with a more limited number of reference terms. Thus, during each iteration of ontology enrichment, a number of conflicting terms found in the extracted expressions may be counted and used to assess performance of extraction by using the ontology. Over subsequent iterations of ontology enrichment, a decreasing rate of conflicting extracted expression may be an indication that an accuracy of the concepts and terms in the ontology (in each targeted language) may be increasing. The conflicting terms may be extracted, together with a few examples and presented to an expert, so that the expert can define whether the conflict is real. If a conflict does not exist, the expert will update the ontology so that the case will no longer be considered a conflict. If the conflict is confirmed, the expert will look for a root cause. For example, a term may be incorrectly mapped to a reference term and/or expression, or an individually extracted term may form part of a multi-word expression. Once the root cause has been identified, the conflict may be resolved in the ontology.

At 812, evaluating the expression extraction may include calculating a total expression recognition rate, over various iterations of ontology enrichment. During each iteration, a total number of expressions extracted (e.g., recognized by the ontology) may be counted. For example, after a first iteration of ontology enrichment, a first number of expressions from the test set may be recognized and extracted (e.g., may match one or more reference terms of the ontology). After a second iteration of ontology enrichment, a second number expressions from the test set may be recognized and extracted. After a third iteration of ontology enrichment, a third number of expressions from the test set may be recognized and extracted. By comparing the number of expressions recognized over the various iterations, a total expression recognition rate may be calculated for each targeted language, and over all targeted languages.

For example, for a selected target language, if 1000 expressions are extracted after the first iteration, 1500 expressions are extracted after the second iteration, and 2000 expressions are extracted after the third iteration, the overall terms extraction rate may be increasing. An increasing overall terms extraction rate may be an indication of an improvement in expression extraction in the targeted language. If, after various iterations of ontology enrichment have been performed, the overall terms extraction rate is unchanged and low, it may be inferred that expression extraction is not improving (e.g., due to a problem in method 700). Alternatively, if after various iterations of method 700 have been performed, the overall terms extraction rate is unchanged and high (e.g., at a plausible maximum overall terms extraction rate), it may be inferred that expression extraction is no longer improving, whereby no further ontology enrichment may be carried out in the selected target language.

Another performance indicator may be a frequency with which expressions from the test set are not recognized (e.g., not mapped to reference terms in the ontology). The less frequent unrecognized terms occur, the better the performance of the ontology at extracting expressions. A maximum frequency threshold may be defined as a target.

It should be appreciated that the performance metrics such as extraction rates described herein are for illustrative purposes, and other types of performance metrics may additionally or alternatively be used to evaluate expression extraction without departing from the scope of this disclosure.

At 814, method 800 includes determining whether expression extraction is improving. For example, if the calculated extraction rate per expression is increasing, it may be inferred that the expression extraction is improving. If the calculated rate of conflicting terms per expression is decreasing, it may be inferred that the expression extraction is improving. If the calculated overall terms extraction rate is increasing, it may be inferred that the expression extraction is improving. In various embodiments, the performance of the ontology may be evaluated separately in each of the target languages, as well as over the set of targeted languages.

If at 814 it is determined that the foreign language expression extraction is improving, method 800 proceeds to 816. At 816, method 800 includes generating a new sample set, and continuing to add terms of the targeted foreign languages to the ontology, for example via method 700. If at 814 it is determined that the expression extraction is no longer improving, method 800 proceeds to 818. At 818, method 800 includes stopping adding terms to the ontology via method 700, and method 800 ends.

Thus, systems and methods are provided herein for processing description field data of a set of DICOM files, using an ontology, to assign a unidimensional type to a medical imaging exam corresponding to the DICOM file. The unidimensional type may be a lexicon entry of an established radiology lexicon, and may subsequently be used by various AI algorithms to selectively perform actions on the medical imaging exam. For example, to ensure that one or more medical imaging exams may be quickly and easily retrieved from a storage system (e.g., a database or archive), the exams may be indexed based on the unidimensional types of the exams. A batch processing system may apply one or more algorithms to one or more medical exams, where the one or more algorithms and/or the one or more medical exams are selected based on the unidimensional types. One or more medical imaging exams may be mapped to a regulatory category of a plurality of regulatory categories, based on the unidimensional types. For example, a unidimensional type may indicate that an exam is a CT exam. As a result of the exam being a CT exam, a dosage of the CT exam may be reported to an appropriate regulatory body. In other embodiments, operational efficiency analytics routines may be performed on one or more medical imaging exams based on the unidimensional types. A sales targeting model may be built based on the unidimensional types, for example to identify a set of relevant options to propose to a prospective client.

Additionally or alternatively, a medical exam may be routed to a radiologist for review via a routing system based on the unidimensional type. For example, a first radiologist may have expertise at reviewing medical imaging exams of a heart of a patient, and a second radiologist may have expertise at reviewing medical imaging exams of a brain of a patient. A first AI algorithm may determine from the unidimensional type of an exam that an anatomical region of interest of the exam is a heart, whereby the routing system may not route the exam to the second radiologist to review, and may route the exam to the first radiologist to review.

When the exam is reviewed by the first radiologist, a second AI algorithm may aid the first radiologist in launching a suitable software application to review the medical imaging exam on a computing device operated by the first radiologist, based on the unidimensional type. For example, the second AI algorithm may be an automated decision assistance service that may determine, from expressions extracted from one or more description fields of a set of DICOM files of the exam, one or more probable options for a suitable application for a radiologist to review the medical exam. The expressions may be extracted using the ontology, which maps terms of the ontology to the extracted expressions. The ontology may also map the extracted expressions to one or more entries of a radiology lexicon, such as the RadLex lexicon. Each entry of the one or more entries may be associated with a candidate application for reviewing the medical exam, whereby a most probable entry discovered in the ontology may indicate a most relevant candidate application.

If a probability of the one or more probable options (e.g., lexicon entries) exceeds a threshold probability, or if a difference between the probability and other probabilities of other lexicon entries being relevant, an automated decision assistance service may automatically select the option. If no probable option is detected, the automated decision assistance service may display the one or more probable options to the user to select from. The user may select a desired option of the one or more probable options in a shorter amount of time than may be taken to select a suitable application from a comprehensive or exhaustive list of available applications, leading to a faster, easier, and more desirable user experience during an exam review workflow.

An advantage of the systems and methods proposed herein is that they do not rely on a standardization of DICOM description fields. Rather, by generating and using the ontology, a wide variety of textual descriptions written in various styles and/or in different languages may be reliably mapped to one or more matching unidimensional types (e.g., lexicon entries). In contrast to other types of machine learning approaches that rely on collecting and processing a large amount of data to “learn” best matches, which may consume a greater amount of expert time for labeling, greater amount of memory and processing resources, the approach described herein uses a computationally efficient process based on text string comparisons that leverages a hierarchical structure of the ontology to quickly generate a set of best potential options. Because the approach described herein relies on less processing power and a smaller amount of stored data, an appropriate option may be automatically selected, or a list of candidate options may be displayed to the user in real time without introducing any delays or lag time, facilitating a faster and more efficient workflow and reducing an amount of user frustration.

In other words, in contrast to other approaches in the domain that use supervised methods involving training sets, the unsupervised methods disclosed herein may generate an ontology that covers DICOM sources from a broad selection of manufacturers, sites, countries, and languages, which would not be possible with the other approaches due to training set limitations in practice. A supervised method would require a huge sample set, with a huge labeling effort to develop ground truth data. Instead, the methods proposed herein focus on collecting vocabulary options from various sources and efficiently presenting the vocabulary options to domain experts with examples providing context.

Thus, the ontology-based approach provides a specific implementation of a solution to generating a preferred option or set of options from a larger general set of options in real time, thus improving the operation of the computing device. Accordingly, the ontology-based approach disclosed herein is not simply directed to any form of storing option data, but instead is specifically directed to a hierarchical ontology, where a broad and comprehensive set of terms may be matched to DICOM description field data by leveraging a structure of the hierarchical ontology. In contrast, alternative recommendation models that rely on high-dimensional statistical algorithms or neural network architectures typically rely on collecting, storing, and manipulating large amounts of data in memory when generating the preferred set of options. As a result, the alternative recommendation models may consume more resources of the computing device, leaving less resources available for other applications running on the computing device. Because of the greater use of resources (e.g., processing power and memory) of the alternative recommendation models with respect to the recommendation model described herein, an amount of time taken by the alternative recommendation models to generate the preferred set of options may not be generated in real time without delays, where the user may have to wait for the preferred set of options to be generated.

Additionally, the ontology-based approach presented herein may be used immediately upon deployment, and does not rely on accumulating an initial amount of data, nor does performance vary over time or increase slowly as information is collected. Further, the performance does not depend on radiologists and other care providers learning and adhering to guidelines for filling in description fields of DICOM files, which may not be enforceable.

Systems and methods are additionally proposed herein for generating and updating the ontology, via a set of procedures that rely on automatic aggregation of data from existing resources and a manual review by human experts. Specifically, a framework is proposed for generating a comprehensive set of terms in a plurality of languages in a manner that ensures an adequate coverage of terms, a high degree of term accuracy, a low rate of conflicting terms, and a minimized labeling effort by experts. As the ontology is updated with new terms, methods for evaluating a coverage and performance of the ontology are presented that do not rely on labeling data or providing ground truth data, which may be costly and time consuming.

The technical effect of automating or facilitating a selection of an appropriate application to review a medical exam using an assistive AI service based on an ontology, is that a time and effort spent by users in navigating various options may be reduced, leading to a faster and more efficient workflow when reviewing the medical exam.

The disclosure also provides support for a method for a medical image processing system, the method comprising: extracting expressions from description fields of a set of Digital Imaging and Communications in Medicine (DICOM) files of a medical imaging exam that match reference terms of an ontology, mapping the matching reference terms of the ontology to one or more lexicon entries of a radiology lexicon, selecting a suitable software application to review the medical imaging exam based on the one or more lexicon entries, opening the suitable software application on a device of the medical image processing system, and displaying the medical imaging exam on a display of the device within the suitable software application. In a first example of the method, the method further comprises: selecting a layout of the suitable software application based on the one or more lexicon entries, and displaying the medical imaging exam in the selected layout of the suitable software application. In a second example of the method, optionally including the first example, the method further comprises: after opening the suitable software application, applying a pre-selected algorithm to the medical imaging exam, the pre-selected algorithm selected prior to opening the suitable software application, based on the one or more lexicon entries, and displaying a result of applying the algorithm on the display of the device within the suitable software application. In a third example of the method, optionally including one or both of the first and second examples, the extracted expressions include at least one of an anatomy, a contrast, a contrast phase, a contrast agent, an acquisition gating, a laterality, a pathology, one or more reconstruction filters, a multi-energy indication, a weighting, a pulse sequence, and one or more options of the medical imaging exam. In a fourth example of the method, optionally including one or more or each of the first through third examples, selecting the suitable application based on the one or more lexicon entries further comprises automatically selecting the suitable application based on a highest priority lexicon entry of the one or more lexicon entries, the highest priority lexicon entry a lexicon entry that most closely matches the one or more extracted expressions. In a fifth example of the method, optionally including one or more or each of the first through fourth examples, selecting the suitable application to review the medical imaging exam based on the one or more lexicon entries further comprises: generating a prioritized list of lexicon entries from the one or more lexicon entries, based on the expressions extracted from the set of DICOM files, eliminating lexicon entries from the prioritized list of lexicon entries that are below a threshold relevance, displaying a list of candidate applications to a user of the medical image processing system based on the prioritized list of lexicon entries, and selecting an application indicated by the user. In a sixth example of the method, optionally including one or more or each of the first through fifth examples, generating the prioritized list of lexicon entries based on the expressions extracted from the set of DICOM files further comprises prioritizing the one or more lexicon entries based on one or more expressions extracted from an exam level description field, a series level description field, and an image series level description field of the set of DICOM files. In a seventh example of the method, optionally including one or more or each of the first through sixth examples, extracting the expressions matching reference terms of the ontology and mapping the matching reference terms of the ontology to the one or more lexicon entries of the radiology lexicon further comprises: combining ontological paths of the reference terms to generate a set of combined concepts of the ontology, mapping the combined concepts of the ontology to the one or more lexicon entries of the radiology lexicon, and ordering the one or more lexicon entries based on the combined concepts. In an eighth example of the method, optionally including one or more or each of the first through seventh examples, combining ontological paths of the reference terms to generate the set of combined concepts of the ontology further comprises combining concepts at an exam level, a protocol level, a series level, and an image group level of the ontology. In a ninth example of the method, optionally including one or more or each of the first through eighth examples, the ontology includes reference terms generated from expressions extracted in one or more target languages, from one or more of: DICOM sources of the one or more target languages, vocabulary found in one or more relevant lexicons, terms found in one or more relevant ontologies, reference terms found in one or more reference databases, and human experts. In a tenth example of the method, optionally including one or more or each of the first through ninth examples, the human experts perform one or more of: resolving conflicting extracted expressions, and adding the resolved expressions to the ontology, adding extracted expressions that are not found in the ontology to the ontology, and for a selected term in the ontology, reviewing a set of extracted expressions in which the selected term is expected and could not be found, and if a similar term to the selected term is present, adding the similar term to the ontology. In a eleventh example of the method, optionally including one or more or each of the first through tenth examples, a performance of the ontology at recognizing new expressions from new DICOM sources is evaluated based on one or more performance metrics, the one or more performance metrics including a per expression extraction rate, a total expression recognition rate, and a rate of conflicting terms per extracted expression. In a twelfth example of the method, optionally including one or more or each of the first through eleventh examples, the lexicon is the RadLex radiology lexicon.

The disclosure also provides support for a system, comprising: a computing device including one or more processors having executable instructions stored in a non-transitory memory that, when executed, cause the one or more processors to: extract description field data from a plurality of Digital Imaging and Communications in Medicine (DICOM) sources, extract a plurality of expressions from the description field data, create an ontology with the extracted expressions, using the ontology, map a new expression extracted from a DICOM file of a medical exam to a unidimensional type of the medical exam, and based on the unidimensional type, configure an application running on the computing device. In a first example of the system, the plurality of DICOM sources includes at least one of: a Picture Archiving and Communication System (PACS), and a log file generated during a performance of a medical imaging exam. In a second example of the system, optionally including the first example, creating the ontology with the extracted expressions further comprises: for each extracted expression, performing at least one of: including a reference term matching the extracted expression in the ontology, including vocabulary related to the extracted expression imported from one or more relevant lexicons into the ontology, including reference terms related to the extracted expression collected from one or more relevant reference databases, and including terms related to the extracted expression collected from one or more reliable ontologies in a domain of the extracted expression. In a third example of the system, optionally including one or both of the first and second examples, further instructions are included in the non-transitory memory that when executed, cause the one or more processors to enrich the ontology with terms from a set of targeted languages, where enriching the ontology further comprises, for each targeted language of the set of targeted languages: translating terms of the ontology with ontology-based translation tools, extracting a set of expressions from description fields of DICOM sources in the targeted languages, using the ontology, displaying translated terms and corresponding expressions of the set of expressions on a display device, for a human expert to manually reconcile, based on input from the human expert, update the ontology with the reconciled translated terms. In a fourth example of the system, optionally including one or more or each of the first through third examples, further instructions are included in the non-transitory memory that when executed, cause the one or more processors to evaluate a performance of the ontology at expression extraction during an extraction of expressions from description fields of a test set of DICOM sources, wherein the performance is evaluated based on at least one of an extraction rate per expression, a total expression recognition rate, and a rate of conflicting terms extracted.

The disclosure also provides support for a method, comprising: extracting one or more expressions from one or more description fields of a plurality of Digital Imaging and Communications in Medicine (DICOM) files, the DICOM files corresponding to a respective plurality of medical imaging exams, for each DICOM file, mapping the expressions extracted from the description fields of the DICOM file to a unidimensional type of a corresponding medical imaging exam, and storing the unidimensional type in a description field of the DICOM file, and performing one or more of: indexing the plurality of DICOM files based on the unidimensional types, applying an algorithm to one or more medical imaging exams of the plurality of medical imaging exams via a batch processing system, based on the unidimensional types, mapping one or more medical imaging exams of the plurality of medical imaging exams to a plurality of regulatory categories, based on the unidimensional types, performing operational efficiency analytics on one or more medical imaging exams of the plurality of medical imaging exams, based on the unidimensional types. In a first example of the method, the method further comprises: at least one of: routing a medical imaging exam of the plurality of medical imaging exams to a radiologist, via a routing system, based on the unidimensional type of the medical imaging exam, and automatically launching a suitable software application for reviewing the medical imaging exam on a computing device operated by a radiologist, based on the unidimensional type.

In another representation, a method for a medical image processing system comprises extracting one or more expressions from one or more description fields of a set of Digital Imaging and Communications in Medicine (DICOM) files of a medical imaging exam; generating an ordered list of lexicon entries matching the extracted expressions; based on the ordered list of lexicon entries, selecting a configuration of a device of the medical image processing system for reviewing the medical imaging exam, from a set of available configuration options; and configuring the device based on the selected configuration.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and expressions set forth herein. Also, as used herein, the examples and embodiments, in all respects, are meant to be illustrative only and should not be construed to be limiting in any manner.

1. A method for a medical image processing system, the method comprising:
extracting expressions from description fields of a set of Digital Imaging and Communications in Medicine (DICOM) files of a medical imaging exam that match reference terms of an ontology;
mapping the matching reference terms of the ontology to one or more lexicon entries of a radiology lexicon;
selecting a suitable software application to review the medical imaging exam based on the one or more lexicon entries;
opening the suitable software application on a device of the medical image processing system; and
displaying the medical imaging exam on a display of the device within the suitable software application.

2. The method of claim 1, further comprising:
selecting a layout of the suitable software application based on the one or more lexicon entries; and
displaying the medical imaging exam in the selected layout of the suitable software application.

3. The method of claim 1, further comprising:
after opening the suitable software application, applying a pre-selected algorithm to the medical imaging exam, the pre-selected algorithm selected prior to opening the suitable software application, based on the one or more lexicon entries; and
displaying a result of applying the algorithm on the display of the device within the suitable software application.

4. The method of claim 1, wherein the extracted expressions include at least one of an anatomy, a contrast, a contrast phase, a contrast agent, an acquisition gating, a laterality, a pathology, one or more reconstruction filters, a multi-energy indication, a weighting, a pulse sequence, and one or more options of the medical imaging exam.

5. The method of claim 1, wherein selecting the suitable application based on the one or more lexicon entries further comprises automatically selecting the suitable application based on a highest priority lexicon entry of the one or more lexicon entries, the highest priority lexicon entry a lexicon entry that most closely matches the one or more extracted expressions.

6. The method of claim 1, wherein selecting the suitable application to review the medical imaging exam based on the one or more lexicon entries further comprises:
generating a prioritized list of lexicon entries from the one or more lexicon entries, based on the expressions extracted from the set of DICOM files;
eliminating lexicon entries from the prioritized list of lexicon entries that are below a threshold relevance;
displaying a list of candidate applications to a user of the medical image processing system based on the prioritized list of lexicon entries; and
selecting an application indicated by the user.

7. The method of claim 6, wherein generating the prioritized list of lexicon entries based on the expressions extracted from the set of DICOM files further comprises prioritizing the one or more lexicon entries based on one or more expressions extracted from an exam level description field, a series level description field, and an image series level description field of the set of DICOM files.

8. The method of claim 1, wherein extracting the expressions matching reference terms of the ontology and mapping the matching reference terms of the ontology to the one or more lexicon entries of the radiology lexicon further comprises:
combining ontological paths of the reference terms to generate a set of combined concepts of the ontology;
mapping the combined concepts of the ontology to the one or more lexicon entries of the radiology lexicon; and
ordering the one or more lexicon entries based on the combined concepts.

9. The method of claim 8, wherein combining ontological paths of the reference terms to generate the set of combined concepts of the ontology further comprises combining concepts at an exam level, a protocol level, a series level, and an image group level of the ontology.

10. The method of claim 1, wherein the ontology includes reference terms generated from expressions extracted in one or more target languages, from one or more of:
DICOM sources of the one or more target languages;
vocabulary found in one or more relevant lexicons;
terms found in one or more relevant ontologies;
reference terms found in one or more reference databases; and
human experts.

11. The method of claim 10, wherein the human experts perform one or more of:
resolving conflicting extracted expressions, and adding the resolved expressions to the ontology;
adding extracted expressions that are not found in the ontology to the ontology; and
for a selected term in the ontology, reviewing a set of extracted expressions in which the selected term is expected and could not be found, and if a similar term to the selected term is present, adding the similar term to the ontology.

12. The method of claim 10, wherein a performance of the ontology at recognizing new expressions from new DICOM sources is evaluated based on one or more performance metrics, the one or more performance metrics including a per expression extraction rate, a total expression recognition rate, and a rate of conflicting terms per extracted expression.

13. The method of claim 1, wherein the lexicon is the RadLex radiology lexicon.

14. A system, comprising:
a computing device including one or more processors having executable instructions stored in a non-transitory memory that, when executed, cause the one or more processors to:
extract description field data from a plurality of Digital Imaging and Communications in Medicine (DICOM) sources;
extract a plurality of expressions from the description field data;
create an ontology with the extracted expressions;
using the ontology, map a new expression extracted from a DICOM file of a medical exam to a unidimensional type of the medical exam, and based on the unidimensional type, configure an application running on the computing device.

15. The system of claim 14, wherein the plurality of DICOM sources includes at least one of:
a Picture Archiving and Communication System (PACS); and
a log file generated during a performance of a medical imaging exam.

16. The system of claim 14, wherein creating the ontology with the extracted expressions further comprises:
for each extracted expression, performing at least one of:
including a reference term matching the extracted expression in the ontology;
including vocabulary related to the extracted expression imported from one or more relevant lexicons into the ontology;
including reference terms related to the extracted expression collected from one or more relevant reference databases; and
including terms related to the extracted expression collected from one or more reliable ontologies in a domain of the extracted expression.

17. The system of claim 16, wherein further instructions are included in the non-transitory memory that when executed, cause the one or more processors to enrich the ontology with terms from a set of targeted languages, where enriching the ontology further comprises, for each targeted language of the set of targeted languages:
translating terms of the ontology with ontology-based translation tools;
extracting a set of expressions from description fields of DICOM sources in the targeted languages, using the ontology;
displaying translated terms and corresponding expressions of the set of expressions on a display device, for a human expert to manually reconcile;
based on input from the human expert, update the ontology with the reconciled translated terms.

18. The system of claim 17, wherein further instructions are included in the non-transitory memory that when executed, cause the one or more processors to evaluate a performance of the ontology at expression extraction during an extraction of expressions from description fields of a test set of DICOM sources, wherein the performance is evaluated based on at least one of an extraction rate per expression, a total expression recognition rate, and a rate of conflicting terms extracted.

19. A method, comprising:
extracting one or more expressions from one or more description fields of a plurality of Digital Imaging and Communications in Medicine (DICOM) files, the DICOM files corresponding to a respective plurality of medical imaging exams;
for each DICOM file, mapping the expressions extracted from the description fields of the DICOM file to a unidimensional type of a corresponding medical imaging exam, and storing the unidimensional type in a description field of the DICOM file; and
performing one or more of:
indexing the plurality of DICOM files based on the unidimensional types;
applying an algorithm to one or more medical imaging exams of the plurality of medical imaging exams via a batch processing system, based on the unidimensional types;
mapping one or more medical imaging exams of the plurality of medical imaging exams to a plurality of regulatory categories, based on the unidimensional types;
performing operational efficiency analytics on one or more medical imaging exams of the plurality of medical imaging exams, based on the unidimensional types.

20. The method of claim 19, further comprising at least one of:
routing a medical imaging exam of the plurality of medical imaging exams to a radiologist, via a routing system, based on the unidimensional type of the medical imaging exam; and
automatically launching a suitable software application for reviewing the medical imaging exam on a computing device operated by a radiologist, based on the unidimensional type.
